(19) United States
(12) Patent Application Publication
(10) Pub. No.: US 2013/0317574 A1
(43) Pub. Date: Nov. 28, 2013

(54) Phototherapy Handpiece

(71) Applicant: Lerner Medical Devices, Inc., Chestnut Hill, MA (US)

(72) Inventors: Zafirios Gourgouliatos, Los Angeles, CA (US); David Chang, Encino, CA (US)

(73) Assignee: Lerner Medical Devices, Inc., Chestnut Hill, MA (US)

(21) Appl. No.: 13/958,354

(22) Filed: Aug. 2, 2013

Related U.S. Application Data
(62) Division of application No. 12/014,442, filed on Jan. 15, 2008, now Pat. No. 8,523,925.
(60) Provisional application No. 60/880,883, filed on Jan. 17, 2007.
(60) Provisional application No. 60/880,812, filed on Jan. 17, 2007.
(60) Provisional application No. 60/880,813, filed on Jan. 17, 2007.
(60) Provisional application No. 60/880,887, filed on Jan. 17, 2007.

(51) Int. Cl. A61N 5/06

(52) U.S. Cl. CPC A61N 5/0624
USPC 607/89; 607/94

(57) ABSTRACT

A phototherapy device is disclosed including: a handpiece; and a beamshaping element housed in the handpiece. The beamshaping element is adapted to receive a beam of light from an ultraviolet light source, modify the shape of the beam, and direct the modified beam to provide illumination of a region of a treatment surface, the region, having a tilable cross section at the treatment surface with an irregularly shaped boundary. In some embodiments, the irregularly shaped boundary is composed of three or more segments, each segment being sinusoid-like. In some embodiments, the beamshaping element includes a screen disposed between the light source and the treatment surface, the screen including a light transmissive with an irregularly shaped boundary, and where the irregularly shaped boundary of the illuminated area corresponds to the irregularly shaped boundary of the light transmissive region.

### CROSS-REFERENCE TO RELATED APPLICATIONS

The present application is a divisional of, and claims priority from, U.S. patent application Ser. No. 12/014,442, entitled “Fiber Optic Phototherapy Device”, filed Jan. 15, 2008 and which claims priority from U.S. Provision Application Ser. No. 60/880,883, U.S. Provisional Application Ser. No. 60/880,812, U.S. Provisional Application Ser. No. 60/880,813, U.S. Provisional Application Ser. No. 60/880,887, each filed Jan. 17, 2007. Each of the above mentioned applications are incorporated by reference herein in their entirety.

### BACKGROUND

This invention relates to treatments for inflammatory diseases of the skin, and specifically to methods for devices and treating ultraviolet light-sensitive dermatoses.

When the disease is localized, targeted devices can direct the light over and around the affected area. A side-effect of this treatment is hyperpigmentation (tanning) of the exposed skin. Typical such devices produce square or round footprints of light on the skin with clearly defined edges. The hyperpigmentation patterns are produced with the exact shape of the footprint of the light that exposed the skin. A typical exposure is in the order of 1 to 30 seconds. The onset of hyperpigmentation is typically within 2 to 12 hours of light exposure. This delayed onset does not give the operator of the device any immediate specific marker to indicate where the skin was exposed. When the lesion to be treated is larger than the footprint of the light pattern, light is deposited on the skin in a tile fashion, one such exposed area next to the previous one. As these exposures are laid on the skin, due to lack of an indication and limitation of accuracy in the placement, the exposed areas either overlap or leave some spaces or gaps of unexposed skin. This results in hyperpigmented areas due to a single exposure or double exposure interlaced with non-tanned areas. These patterns do not look natural and are unsightly. As mentioned above, the onset of the hyperpigmentation is within the day of exposure to light. This condition lasts from a few days to sometimes 2-3 months, depending on the intensity of the exposure and the skin type of the patient.

To overcome this problems operators place dots over the skin with an ink marker to guide the placement or attach a small rubber stamp with ink to the handpiece that leaves a mark every time an area is exposed. Both of them do not provide a satisfactory solution.

The current invention offers a solution to this problem with the use of tilable patterns that have edges comprising of sinusoid-like lines. These patterns, when tiled next to each other, if they are not perfectly aligned, produce hyperpigmentation patterns that have curved edges and the hyperpigmented areas from the single exposure or double exposure as well as the non-tanned areas resemble pattern that naturally occur on human skin. These light induced patterns can be oval, round, or have the forms of curved lines. On the contrary, hyperpigmentation and non pigmented areas resulting from exposures produced by prior devices that have straight edges (straight lines in the sense when a transparent flat piece of glass is pressed on the skin so that the area blow it becomes flat).

In addition, patterns that have perforated “postal stamp” type edges, comprising of small semicircle indentations and extensions, produce more natural looking patterns in the double exposed, exposed or unexposed areas of the skin.

When such devices are combined with photosensitizers, the result is similar and often more pronounced.

### SUMMARY OF THE INVENTION

The present invention includes novel therapy devices for effective treatment of inflammatory dermatoses such as psoriasis. These devices comprise an optical source including means for generating ultraviolet (UV) light (radiation) in a predetermined spectral range, and a light delivery apparatus that produces a pattern of light on the skin surface that has sinusoid-like edges or perforated “postal stamp” type edges comprising of small semicircle indentations and extensions.

### BRIEF DESCRIPTION OF THE FIGURES

The foregoing and other objects of this invention, the various features thereof, as well as the invention itself, may be more fully understood from the following description, when read together with the accompanying drawings in which:

FIG. 1 is a perspective view showing an exemplary phototherapy handpiece of the invention;

FIG. 2 is a perspective view with the enclosure cover removed, showing the parts of the exemplary phototherapy device of the invention;

FIG. 3 shows two exemplary patterns produced by the device; and

FIG. 4 shows one exemplary pattern with perforated edges produced by the device.

### DETAILED DESCRIPTION OF THE DRAWINGS

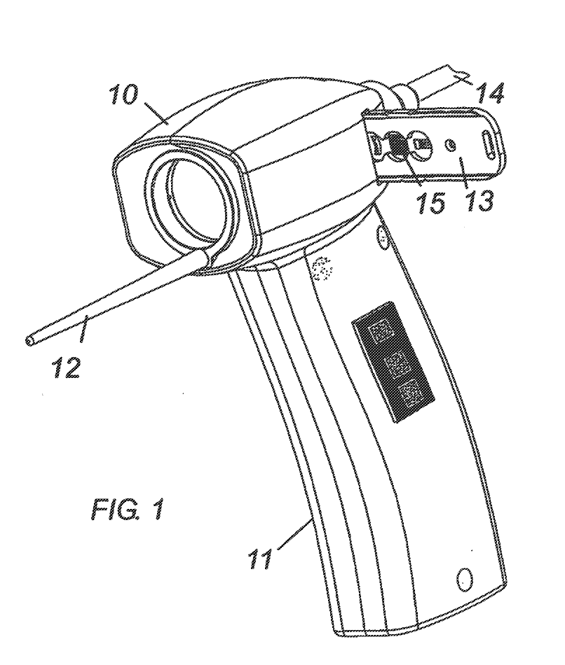

Therapeutic device 10 embodying the invention is shown in FIG. 1, and includes a light delivery apparatus including body member 11 with a handle for convenient use, a distance gage 12 and a plurality of light transporting and shaping elements such as lenses and a light transporting waveguide 14 detachably affixed therefrom. The light goes through one selected screen 15 affixed on a support 13 and the light, having the pattern of the screen is projected onto the skin with the light transporting and shaping elements.

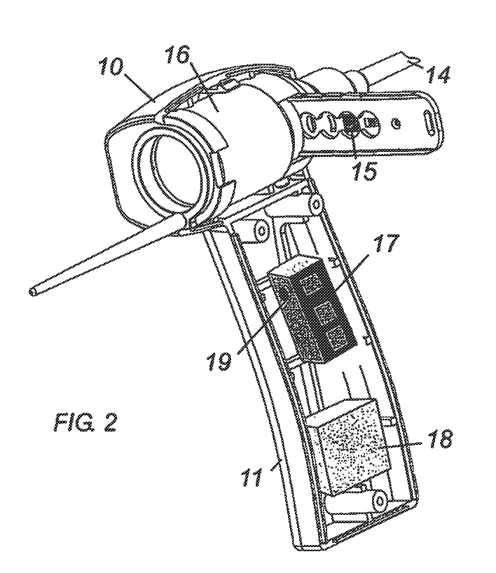

As shown in detail in FIG. 2, body member 11 contains the light transporting and shaping elements 16 that can made out of a single lens or an assembly of lenses affixed in proximity to the a light transporting waveguide 14 and screen 15. Preferably, body member 11 has substantially no UV-transferring abilities, and is formed of a molded resinous material, such as plastic, rubber, and the like.

Body member 11 can include controls such as triggers for light activation as well as display and input elements 17, an auditory signal transducer 19 for operator warnings, etc. In addition it can have a cord that wraps around the wrist of the operator to prevent it from falling to the ground accidentally.

In some embodiments, the power supply and control module 18 may instead be inside the body member. The electrical power source can also be embodied in the body member in the form of batteries, preferably rechargeable. The activation of the device can be external with a switch, attached to the control module, or connected with a cable, or in the form of a foot pedal or with wireless communication.

The distance gage can be combined with a sensor to detect contact between the tip of the gage and the skin of the patient. This will provide additional safety by preventing the device to be activated unless the tip is in contact with the skin.

The light power can be provided by, but not limited to, laser, excimer laser, excimer gas discharge lamps, Light Emitting Diode (LED), or lamp with high pressure, short arc or other type. The light producing element can be inside or external to the body member 11 with the control module connected with electrical cable to the body member or embodied 18 in the body member enclosure as mentioned above.

In some embodiments, the optical power of a different wavelength can be inside or outside the enclosure and converted to light in the UV-B spectrum by a wavelength converter consisting of photon excitable material. In the case that the light is produced externally to the handpiece, it will be transported to the handpiece with a light guide.

If the light of the embodiment described in the last paragraph is produced outside the handpiece, the light power will be transported to the hand piece vial a flexible lightguide or fiber optic. The flexible lightguide can be of the type of liquid filled pipe. The fiber optic can be of quartz or fused silica material with glass or polymer cladding.

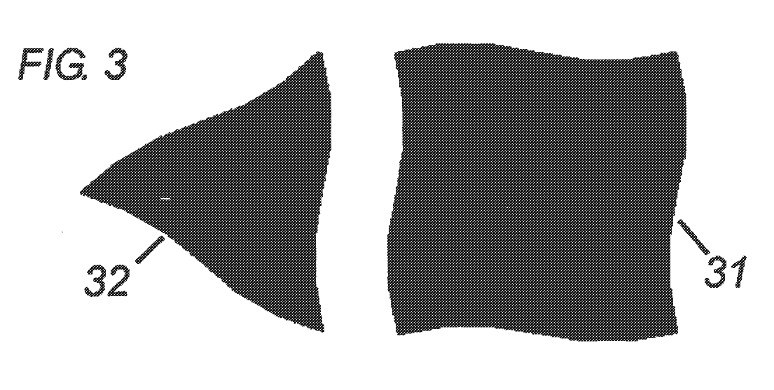

Light pattern producing screens can be retractable and interchangeable. One such embodiment is shown in FIG. 3.

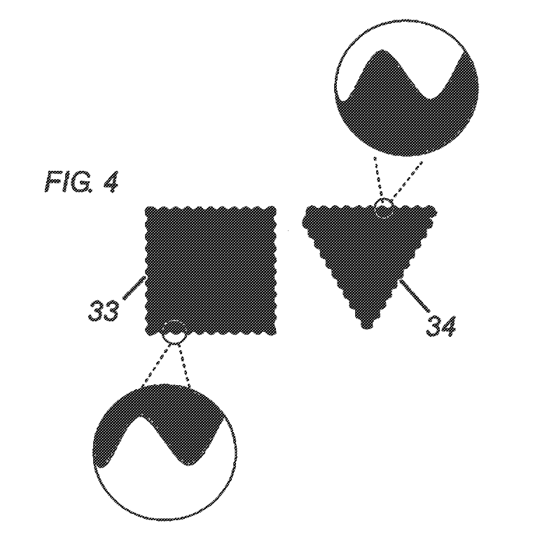

Various patterns are shown in FIGS. 3 and 4. The square with the sinusoid-like edges 31, triangle with similar edges 32 and square 33 or triangle 34 with perforated, “postal stamp” type edges will give the best results, depending on the area of the skin that is treated.

The distance gage is detachably attached to body member, made of hardy material, either for single use or autoclavable or able to be gas sterilized.

The advantage of this device is that localized areas of the skin may be treated without exposing the entire body to photosensitizers and/or to UV light.

In a preferred embodiment, the light producing elements can emit light in the spectral range of 280-320 nm, preferably in the 308-320 nm for dermatoses that respond to UV-B light or Narrow band UV-B light. Alternatively they can emit light in the 320-380 nm for dermatoses that respond to UV-A light, as well as in other spectral bands of visible or infrared light for dermatoses or skin conditions that respond to specific wavelengths.

Preferred embodiments of the method of treating an inflammatory dermatosis using the aforementioned device are as follows.

For UV-B phototherapeutic treatment, exposure with prescribed dose will be tiled on the skin until affected area is treated. Depending on the treatment protocol, exposure can start with approximately one minimum erythema dose (MED), or a multiple of it. Subsequent treatment times would increase if needed and as tolerated by the patient.

In practice, the delivered exposure dose (fluence) needs to be controlled to within about 40% absolute. Both short-tem and long-term output stability, including solarization of optical elements, are considerations affecting dosimetry. If the source output is stable (e.g., less than 10% variation of UV irradiance) after a short warm-up period, over the duration of one treatment (typically a few seconds per exposure), then a timer type of device to control delivered dose based on a measured irradiance is appropriate. If the output is unstable, an integrating dosimeter is required. The ideal system would be stable, might require the user to point the output onto a detector which measures irradiance appropriately, and then enter the desired dose in J/cm².

Of course, certain areas of the skin such as the nails could also be treated as above.

The invention may be embodied in other specific forms without departing from the spirit or essential characteristics thereof. The present embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein.

1-32. (canceled)

33. A light delivery apparatus for use in phototherapy comprising:
a handpiece; and
a beamshaping element housed in the handpiece;
wherein the beamshaping element is adapted to
receive a beam of light from an ultraviolet light source,
modify the shape of the beam, whereby the beam has a cross-section with an irregularly shaped, tilable boundary comprising three or more sinusoid-like edges, and
direct the modified beam to provide illumination of a region of a treatment surface.

34. The apparatus of claim 33, wherein the beamshaping element comprises a screen disposed between the light source and the treatment surface, said screen comprising a light transmissive region with an irregularly shaped boundary, and wherein the irregularly shaped boundary of the illuminated area corresponds to the irregularly shaped boundary of the light transmissive region.

35. The apparatus of claim 34, further comprising a light guide configured to deliver light from the ultraviolet source to the handpiece.

36. The apparatus of claim 35, further comprising an ultraviolet light source.

37. The apparatus of claim 36, wherein the light source is housed in the handpiece.

38. The apparatus of claim 34, wherein the light source comprises at least one from the group consisting of: a light emitting diode, a lamp, a laser, an excimer laser, a diode laser, and an excimer gas discharge lamp.

39. The apparatus of claim 36, wherein the ultraviolet light source comprises:
a first source of light at a first wavelength; and
a wavelength converter adapted to convert at least a portion of the light from the first source to provide a beam of ultraviolet light.

40. The apparatus of claim 36, wherein the light source has a spectral output within the range of 280 nm to 320 nm.

41. The apparatus of claim 39, wherein the light source has a spectral output within the range of 308 nm to 320 nm.

42. The apparatus of claim 36, wherein the light source has a spectral output within the range of 320 nm to 380 nm.

43. The apparatus of claim 34, wherein the irregularly shaped boundary is described by one of the group consisting of: a square with curved edges, a triangle with curved edges, a square with postage stamp type edges, a triangle with postage stamp type edges, and a polygon with one or more curved, or postage stamp type edges.

44. The apparatus of claim 36, further comprising a control module in communication with the beam shaping element or light source, said control module adapted to selectively adjust the duration or intensity of the illumination.

45. The apparatus of claim 44, further comprising:
a distance gauge affixed to the handpiece and extending between an end proximal to the handpiece and an end distal to the handpiece.

46. The apparatus of claim 45, comprising a sensor adapted to sense contact of the distal end of the distance gauge with a treatment surface and generate a contact signal indicative of said contact.

47. The apparatus of claim 45, wherein the distance gauge is detachably affixed to the handpiece.

48. The apparatus of claim 46, wherein the control module is adapted in response to the contact signal to inhibit the illumination of the treatment surface when the sensor does not indicate contact of the distal end of the distance gauge to the treatment surface.

49. The apparatus of claim 36, further comprising a power supply adapted to supply power to the ultraviolet light source.

50. The apparatus of claim 49, wherein the power supply is housed in the handpiece.

51. The apparatus of claim 34, wherein the beam shaping element comprises one or more lenses.

52. The apparatus of claim 51, wherein the one or more lenses are adapted to direct light transmitted through the screen.

53. The apparatus of claim 34, further comprising a support comprising the screen, wherein the support is retractably house in the handpiece.

54. A light delivery apparatus for use in phototherapy, comprising:
a handpiece comprising a beamshaping element, said beamshaping element adapted to receive a beam of light from an ultraviolet light source, modify the shape of the beam, whereby the beam has a cross-section with an irregularly shaped, tilable boundary comprising three or more sinusoid-like edges; and direct the modified beam to provide illumination of a region of a treatment surface; and
a plurality of screens adapted to be interchangeably received by the beam shaping element at a position disposed between the light source and the treatment surface, each of said screens comprising a light transmissive region with an irregularly shaped boundary, wherein the irregularly shaped boundary of the illuminated area corresponds to the irregularly shaped boundary of the light transmissive region;
wherein the irregularly shaped boundary is composed of three or more segments, each segment being sinusoid-like; and
wherein the shape of the boundary of the light transmissive region of each screen varies from screen to screen.

55. The system of claim 54, further comprising a support retractably housed in the handpiece, said support comprising one or more of the plurality of screens.
